(19) United States
(12) Patent Application Publication
(10) Pub. No.: US 2006/0229515 A1
(43) Pub. Date: Oct. 12, 2006

(54) Fiber optic evaluation of tissue modification

(75) Inventors: Shiva S. Sharareh, Irwindale, CA (US); Stavros Demos, Livermore, CA (US)

Correspondence Address: Michael C. Staggs;Attorney for Applicants, Lawrence Livermore National Laboratory, P.O.Box 808, L-703, Livermore, CA, 94551, US

(73) Assignee: The Regents of the University of California

(21) Appl. No.: 11/414,009

(22) Filed: Apr. 27, 2006

Related U.S. Application Data
(63) Continuation-in-part of application No. 11/281,853, filed on Nov. 17, 2005.
(60) Provisional application No. 60/629,166, filed on Nov. 17, 2004.

(51) Int. Cl. A61B 6/00

(52) U.S. Cl. 600/476

(57) ABSTRACT

An optical method and apparatus is utilized to evaluate the presence of tissue modification, in particular, to evaluate tissue ablation using light scattering spectroscopy realized via optical fiber(s). Such a technique allows for detection of the presence of tissue modification and provides depth information, such as, for example, depth of an ablated lesion. The method and apparatus as described herein can be used for in-vivo, real-time monitoring during predetermined procedures, such as, cardiac tissue ablation for therapeutic reasons.

### RELATED APPLICATION

This application is a Continuation-in-Part of application Ser. No. 10/260,141 entitled “Fiber-Optic Evaluation of Cardiac Tissue Ablation,” filed Nov. 17, 2005, which claims priority from U.S. Provisional Patent Application No. 60/629,166, also entitled “Fiber-Optic Evaluation of Cardiac Tissue Ablation,” filed on Nov. 17, 2004, both of which are incorporated by reference in its entirety.

The United States Government has rights in this invention pursuant to Contract No. W-7405-ENG-48 between the United States Department of Energy and the University of California for the operation of Lawrence Livermore National Laboratory.

### BACKGROUND OF THE INVENTION

1. Field of the Invention

The present invention relates to a medical diagnostic. More particularly, the present invention relates to optical interrogation configurations for investigating tissue modification in real-time during medical procedures.

2. Description of Related Art

There are a number of conditions that can be addressed via the destruction of tissue regions to achieve a beneficial result for a patient. Such tissue destruction is typically achieved by subjecting the tissue to conditions outside the environmental profile needed to sustain the tissue alive. As an example, cardiac tissue ablation electrode catheters that can be inserted percutaneously under local anaesthesia into a femoral, brachial, subclavian, or internal jugular vein and positioned in the heart using techniques developed by those skilled in the field is performed to address cardiac arrhythmias, e.g., fibrillation.

In general, ablation systems include an ablation catheter or similar probe having an energy-emitting element. The energy-emitting element delivers energy forming a lesion in the targeted tissue. Typical elements include a microwave ablation element, a cryogenic ablation element, a thermal ablation element, a light-emitting ablation element, an ultrasound transducer, and/or a radio frequency ablation element. The ablation catheter may be adapted to form a variety of lesions such as linear lesions or a circumferential lesion. The element is connected to an energy source that can be varied to control the formation of the lesion.

While various types of ablation catheters for various therapeutic procedures currently exist, catheter ablation of cardiac tissue in particular, is typically performed using radiofrequency energy delivered as a continuous, unmodulated, sinusoidal waveform having a frequency of about 500 kilo-cycles per second. The majority of such systems utilizes the temperature of the ablation electrode to monitor tissue modification, such as lesion formation, and automatically adjusts power output to achieve a targeted electrode temperature. Knowledge of the electrode temperature at a particular ablation site is useful in determining whether the application of radiofrequency produced the desired ablation but it is not sufficient to accurately predict the dimensions of the lesion created, especially its depth.

Thermal injury is the principal mechanism of tissue destruction during radiofrequency catheter ablation procedures. Elevation of catheter temperature can also result in non-desirable conditions, such as, coagulation of the blood. The development of a coagulum, which can represent a hazard to the patient (i.e., via stroke), results in a rapid increase in impedance which leads to a dramatic decrease in current density, thereby limiting further lesion growth. Moreover, the ablation process can also cause undesirable charring of the tissue and can generate evaporate water in the blood and tissue leading to bursts of microbubbles (i.e., steam pops) during the ablation procedure, which are the result of deposition of energy at a faster than desired rate. Automatic adjustment of power output using closed loop temperature control has been shown to reduce the incidence of coagulum development, steam pops, and undesired charring, which may also facilitate catheter ablation by, for example, reducing the number of times the catheter has to be withdrawn from the body-to have a coagulum and charring material removed from the electrode tip.

Despite improvement in the current technologies, no real-time feedback system and method regarding the condition (e.g., the creation of lesions in the lateral and axial dimensions) of the treatment site in addition to the formation of coagulum, steam pops, and charring during catheter ablation within the body is currently available.

Accordingly, a need exists for methods and instrumentation to primarily provide real-time feedback during such procedures as to determine lesion formation, physical dimension, the formation of charred tissue, steam pops, and coagulated blood around a predetermined ablation catheter or endoscopic instrument for any given procedure, medical or otherwise. The present invention is directed to such a need.

### SUMMARY OF THE INVENTION

Accordingly, the present invention is directed to a spectroscopic method for real-time examination of biological tissue that includes: deploying a diagnostic and/or treatment tool on, in, or near a predetermined tissue site; directing the diagnostic and/or treatment tool to modify one or more tissue components located at the tissue site; providing one or more predetermined optical conduits adapted to direct an interrogation radiation source at the tissue site and one or more predetermined optical conduits adapted to receive an induced predetermined backscattered radiation from the tissue site resulting from the directed interrogation radiation; and measuring before, during, or after the modification step, one or more NIR elastic light scattering spectra resulting from the induced NIR backscattered radiation to assess in real-time, a lesion formation, a depth of penetration of the lesion, a cross-sectional area of the lesion in the tissue, recognition of charring, recognition of the formation of coagulum, differentiation of ablated tissue from healthy tissue, and/or recognition of evaporate water in the blood and tissue leading to steam pops.

Another aspect of the present invention provides a treatment and/or diagnostic tool that can be configured with optical fiber arrangements to provide real-time analysis of lesion formations, depth of penetration of a lesion, a cross-sectional area of a lesion in the tissue, recognition of charring, recognition of the formation of coagulum, differentiation of ablated tissue from healthy tissue, and/or recognition of evaporate water in the blood and tissue leading to steam pops.

Accordingly, the present invention provides optical arrangements and methods, capable of directing predetermined spectral radiation and capable of providing received and analyzed spectral information for the determination and quantification of normal or modified tissue. Applications include assessment of tissue parameters during cardiac ablation as well as assessment of tissue properties such as the formation of plaque, artery thickness, and scar tissue.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1(a) shows a simplified diagram of a fiber optic evaluation system of the present invention.

FIG. 1(b) shows another example fiber optic evaluation arrangement of the present invention.

FIG. 1(c) shows another beneficial fiber optic evaluation arrangement of the present invention.

FIG. 2(a) shows a generic fiber optic implementation within a treatment catheter.

FIG. 2(b) shows a beneficial modification of the fiber arrangement within a treatment catheter.

FIG. 3(a) shows real-time detection of intensity changes during catheter ablation treatment.

FIG. 3(b) shows a real-time monitoring spectrum for 5 different ablation depths.

FIG. 4 illustrates the relationship between depth and spectral profile using as a marker, the slope of the profile after a linear fit of the profile between 730 nm and 900 nm.

FIG. 5(a) illustrates the real-time detection of coagulum formation during catheter ablation treatment from the characteristic changes in the detected spectral profile.

FIG. 5(b) illustrates the real-time detection of charring during catheter ablation treatment.

### DETAILED DESCRIPTION OF THE INVENTION

Referring now to the drawings, specific embodiments of the invention are shown. The detailed description of the specific embodiments, together with the general description of the invention, serves to explain the principles of the invention.

Unless otherwise indicated, numbers expressing quantities of ingredients, constituents, reaction conditions and so forth used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical value, however, inherently contain certain errors necessarily resulting from the standard deviation found in their respective testing measurements.

General Description

The apparatus and methods, as disclosed herein, allow real-time qualification and quantification of tissue components, often during catheter ablation treatment of predetermined tissue components, such as the heart. By utilizing the disclosed techniques of the present invention, lesion formation, depth of penetration of the lesion, cross-sectional area of the lesion in the tissue, recognition of charring, recognition of the formation of coagulum, differentiation of ablated tissue from healthy tissue, and recognition of evaporate water in the blood and tissue leading to microbubbles (i.e., steam pop formation) is beneficially enabled.

Beneficial ablation catheter embodiments of the present invention are often configured with an optical conduit, i.e., optical fibers or fiber bundles disposed within the catheter from the proximal to about the distal end. The collection and detection system can include any of the optical means for collecting, e.g., refractive and reflective optics, filtering, e.g., notch filters, band-pass filters, edge filters, etc. and/or spectrally dispersing (e.g., using for example, predetermined spectrographs) received polarized and often unpolarized induced spectra so as to capture, and thus best quantify and qualify the spectral information of tissue components often undergoing modification. The detectors themselves often include charged coupled devices (CCDs), (e.g., front and back illuminated CCDs, liquid nitrogen cooled CCDs, on-chip amplification CCDs) but can also include photodiodes, photomultipliers, multi-channel spectral analyzers, two-dimensional array detectors, multi-array detectors, or any equivalent means to provide acquisition, often digitized acquisition, of one or more spectra.

During tissue modification, such as, but not limited to, thermal or cryo tissue ablation, an operator can obtain real-time feedback information about the site undergoing modification. By monitoring the intensity (often up to or greater than a two fold change in peak intensity) of NIR received elastic light scattered spectra between about 600 nm and about 1500 nm, an operator can detect the onset as well as track the progress of tissue ablation.

Moreover, the relative intensity of the red-shifted component of the spectral profile increases as a function of the depth of ablation in time and deposited thermal energy. Thus, the changes in the spectral profile can be used to evaluate the depth of the lesion. In a simplified method of analysis of the changes in a spectral profile, an operator can use the slope of received spectra (i.e., defined by ratios of predetermined spectral bands of received spectra, such as the ratio of the 730 nm over the 910 nm part of the spectrum of received red-shifted spectra) for depth profiling using appropriate calibration methods known to those skilled in the art. Such a beneficial arrangement enables a user to extrapolate ablation depths past the point of directed illumination wavelength penetration depths. Other aspects of the received spectra can be utilized to monitor charring, coagulum, and/or steam pop formation due to observed characteristic changes as shown below in the present invention.

Thus, from such information, operators or automatic software driven directions through closed loop operations can determined the exposure time and/or terminate a procedure, or increase or decrease the energy delivered to the site as required for a desired effect (e.g., for greater lesion formation at a desired depth), or detect the formation of charring, coagulum, or the formation of steam pops or determine whether an application of ablation energy failed to reach a desired tissue modification.

Accordingly, the present invention provides methods and apparatus for rapid, in-vivo detection and evaluation of modified tissue components. In particular, the present invention provides elastic Near-infrared (NIR) light (i.e., elastic light scattered spectra between about 600 nm and about 1500 nm) scattering inspection techniques and optical arrangements, often configured with ablation catheter embodiments, as known and utilized by those skilled in the art, to monitor in real-time, human tissue components undergoing tissue modification or for simple probe analysis. Beneficial aspects of utilizing NIR as an analysis means when coupled to probes as discussed herein, include, but are not limited to:
penetration depths of up to about a few centimeters inside targeted tissue components;
minimized influence by blood due to low absorption;
incorporated inexpensive technology;
no danger to the operator or the patient;
non-invasively provided information from the surface as well as below the surface of the tissue;
fiber optic methods that can be easily incorporated in various devises to direct predetermined illumination spectral bands as well as receive real-time feedback from remote locations undergoing treatment.
   Specific Description

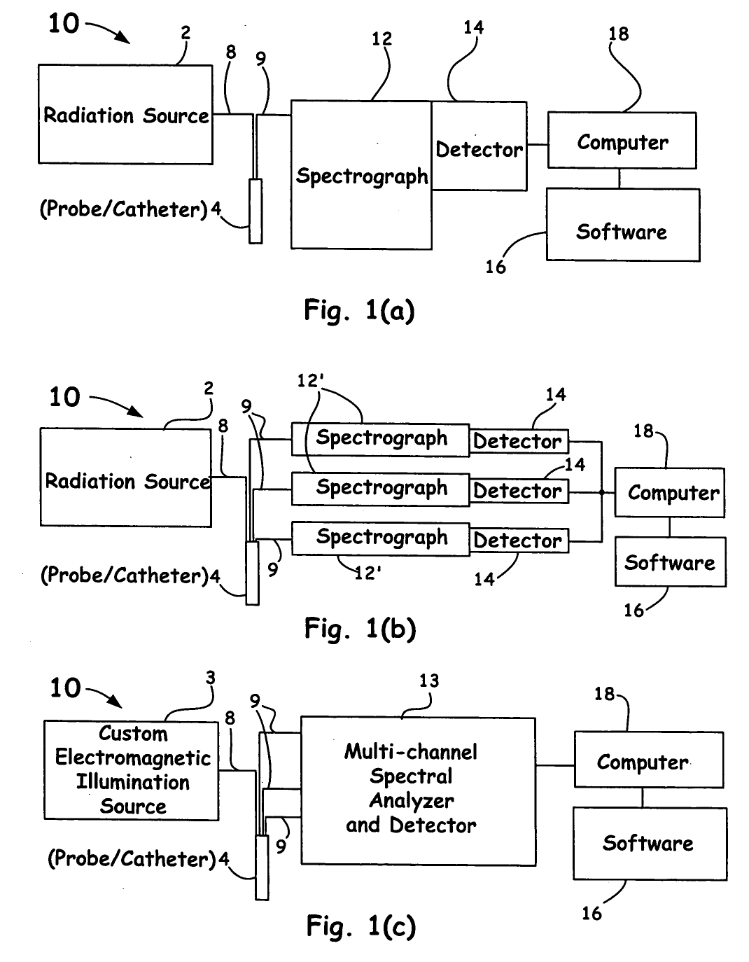

Turning now to the drawings, diagrams that illustrate exemplary basic embodiments of systems constructed in accordance with the present invention are shown in FIGS. 1(a)-1(c). Such systems, designated generally by the reference numeral 10, is most often automated by an analysis means, such as software program 16, residing on a control analysis means 18 (e.g., a computer, firmware (ROM's, EPROM's) and integrated computational, storage, etc., circuit means, such as, but not limited to, large scale Integrated Circuits LSIC (LSIC), very large scale Integrated Circuits (VLSIC), and field-programmable gate arrays (FPGA's)), which is operably coupled to each component in system 10 by predetermined wireless and or hard communication lines (not shown) such as, USB or RS232 cables. Such software means, firmware means, and other integrated circuit means can provide the filtering, storage and computational manipulations that is desired for the present application. Such communication lines can be constructed and arranged to allow for the exchange of information between analysis means 18 and the system components as shown in FIGS. 1(a)-1(c) to effect operation in a prescribed sequence at the direction of an operator or a predetermined set of programmed instructions to transfer spectral information to analysis means 16 for storage and immediate analysis during operational procedures.

System 10, also includes an electromagnetic radiation source 2, as shown in FIG. 1(a) and FIG. 1(b), for illumination of targeted tissue components. Because the present invention utilizes NIR light scattering and in some arrangements polarized NIR light scattering techniques to determine and quantify tissue modification of, for example, an ablated region of a heart, such a radiation source often includes emission wavelengths of greater than about 250, often a monochromatic laser light source operating at wavelengths of up to about 1500 nm, but most often from about 600 nm to about 970 nm in wavelengths, or from any non-coherent, broadband and/or a coherent source capable of being integrated into the present invention so as to delineate differences in absorption and scattering in human tissue components and to provide mean photon penetration depths of up to about 1 cm. In particular, such sources can include broadband sources (e.g., incandescent lamps, arc lamps, wide-band LEDs), narrow-band spectrally stable light emitting diodes (LEDs), narrow-band fluorescence sources, tunable optical sources (e.g., an optical parametric oscillator, dye lasers, or a Xenon source coupled with a computer controlled monochrometer), narrow-band stable lasers, tripled Nd:Yag systems, etc., all of which are capable of emitting predetermined filtered or otherwise spectral bands to interact with desired tissue components (not shown) so as to induce the desired NIR scattered spectral information.

Such radiation sources 2, can be configured with probe/catheter 4 via one or more operably coupled optical conduits, e.g., hollow waveguides, light guides, fiber(s) 8, etc., often large core optical fibers (i.e., multimode fibers) or fibers suitably designed with predetermined fiber indices and dopant profiles, tapered fiber ends and/or special cavity configurations (e.g., bend loss loops), etc. for maintaining polarization properties for predetermined applications, such as when desiring elastic differential light scattering information from a targeted tissue component.

Such differential light scattering techniques that can also be utilized in the present invention is similarly discussed and disclosed in U.S. Pat. No. 7,016,717 B2, titled “Near-Infrared Spectroscopic Tissue Imaging In Medical Applications,” by Demos et al., the disclosure of which is herein incorporated by reference in its entirety. Accordingly, cross-polarization and normalization analysis coupled with inter-spectra operations, such as, but not limited to, subtraction between one or more predetermined received spectra or division between predetermined spectral bands of a received spectra provide information as to the tissue properties resulting from one or more respective probe illumination wavelengths. In addition, the incorporated NIR elastic light scattering intensity measurements of modified tissue components during treatment procedures, often during catheter ablation treatment, using predetermined wavelength cross-polarized light spectrometry, also can provide information for lesion mapping, lesion formation determination and quantification.

As another beneficial arrangement, a custom electromagnetic radiation source(s) 3, as generically shown in FIG. 1(c), can be configured along with or in substitution of a broadband source, as discussed above, to provide directed desired power levels of at least about 1 μW in one or more spectral bands/wavelengths of up to about 1500 nm, but most often from about 600 nm to about 970 nm in wavelengths, to about the distal end of the probe/catheter 4 via optical fiber(s) 8. Example custom electromagnetic radiation source(s) 3 can include, but are not limited to, one or more compact substantially coherent commercial diode lasers arranged with the desired spectral bandwidth, power levels, and geometries, for illumination of predetermined tissue components to induce NIR elastic scattered radiation between about 600 nm and about 1500 nm.

Upon illumination of desired tissue components from about the distal end of probe/catheter 4, via optical fiber(s) 8, one or more additionally optical fibers 9 (e.g., one or more large core multimode fibers, polarization maintaining fibers, etc.) are additionally configured to collect NIR elastic backscattered information about the distal end of probe/catheter 4 induced by light source 2 or light source 3, as shown in FIGS. 1(a)-(c).

It is to be appreciated that the optical fiber embodiments (i.e., fibers shown by reference numerals 8 and 9, as shown in FIGS. 1(a)-(c)), and as disclosed herein, can be configured with any probe, such as, a hand-held probe for topical investigation of tissue modification and it is to be noted that such fiber embodiments can be adapted with enhancing optical elements with respect to its ability to deliver and collect light to and from multiple locations in order to accommodate tissue interrogation of catheter positions from about a normal (i.e., 90 degrees) to about a parallel configuration (i.e., 90 degrees from the normal) with the interrogated tissue. Such enhancing optical elements can include, micro-lenses, mirrors, graded-index lenses, diffractive optical elements and other performance enhancing elements as known in the art.

As another beneficial arrangement, optical fiber configurations can be arranged with a probe, such as, for example, any of the rigid scopes utilized during endoscopic surgery and/or any of the flexible scopes generally reserved for diagnostic examinations and biopsies of tubular body cavities and/or structures, e.g., the upper intestinal tract being examined with a gastroscope. Although the optical configurations of the present invention can be adapted with any of the treatment and/or diagnostic tools currently in the field, most often, however, the optical fiber embodiments of the present invention entail coupling with any of the surgical ablation devices utilized for treatment of tissue components, such as, tissue components of the heart, prostate, and liver. Exemplary variations of such surgical ablation devices are described in U.S. Pat. No. 6,522,930 the disclosure of which is incorporated by reference and as discussed in application Ser. No. 10/260,141 entitled “Fiber-Optic Evaluation of Cardiac Tissue Ablation,” also incorporated by reference in its entirety.

The desired scattered radiation from tissue components as directed by optical conduits (e.g., optical fibers 9) can be filtered through one or more optical components (not shown), such as, edge filters, band-pass filters, polarization filters, prisms, and/or notch filters, etc. Beneficial embodiments, however, can simply include a single spectrograph 12, as shown in FIG. 1(a), or, one or more spectrographs 12′, as shown in FIG. 1(b), (three are shown for simplicity), such as when utilizing catheter embodiments that are arranged to provide information to predetermined spectrographs for angular detailed information of a treated site.

Such spectrographs (note: spectrographs, spectrometers, and spectrum analyzers are used interchangeably) often include optical spectrum analyzers, such as, two-dimensional spectrum analyzers, single or single curved line spectrum analyzers, (i.e., a multi-channel spectrum analyzer 13), to provide, for example, screened cross-section spectroscopic information of a treated or a pre-treatment site. Fourier transform imaging spectrometers or other such devices to allow desired bands and/or polarized components of electromagnetic radiation from tissue components (not shown) can also be used to disperse and analyze received spectra.

A detector 14, as shown in FIGS. 1(a), or a plurality of detectors, as shown in FIG. 1(b), (a detector is not shown in FIG. 1(c) for simplicity) and as discussed above, often include charged coupled devices (CCDs), (e.g., front and back illuminated CCDs, liquid nitrogen cooled CCDs, on-chip amplification CCDs) but can also include photodiodes, photomultipliers, two-dimensional array detectors, a multi-array detector, or any equivalent means of acquisition, often digitized acquisition, of one or more spectra.

The control system software 16, which can be beneficially automated, often includes a graphical user interface (GUI) configured from Visual Basic, MATLAB®, LabVIEW®, Visual C++, or any programmable language or specialized software programming environment to enable ease of operation when performing probe analysis, but more often, probe analysis during catheter ablation treatment of predetermined sites, such as, in predetermined sites of the heart. LabVIEW® and/or MATLAB® in particular, is specifically tailored to the development of instrument control applications and facilitates rapid user interface creation and is particularly beneficial as an application to be utilized as a specialized software embodiment when desired. The received one or more spectra are then captured and stored by analysis means 18 for storage and immediate analysis during operational procedures, which then allows an operator to effect desired changes to, for example, the time of the treatment procedure.

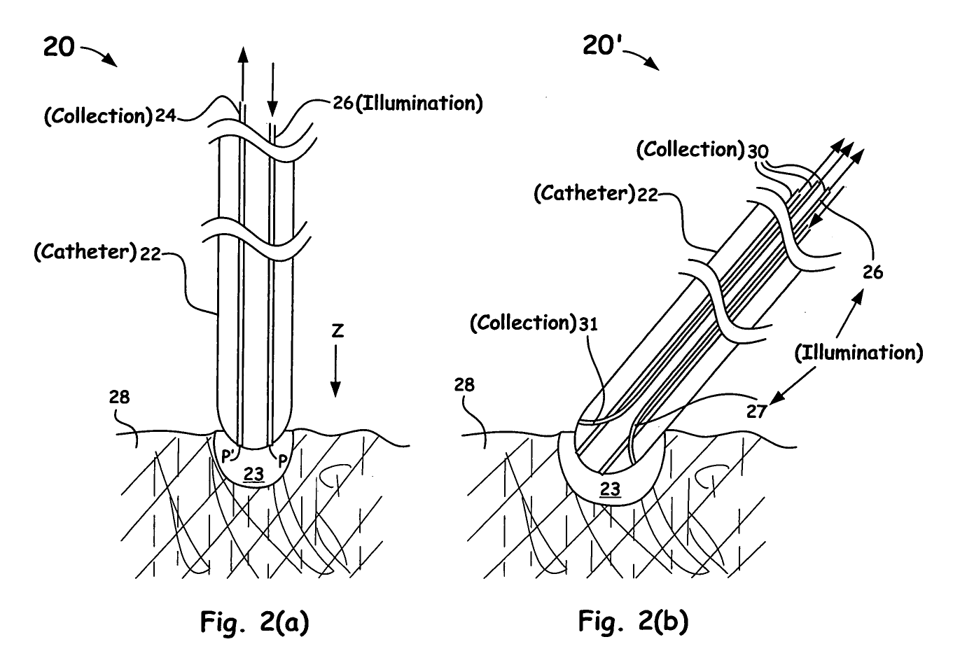

FIG. 2(a) shows a basic catheter embodiment of the present invention, generally designated as reference numeral 20, for real time monitoring of, for example, tissue ablation during treatment of predetermined organs, such as, but not limited to, the liver, prostate, and heart (e.g., a cardiac ablation catheter (e.g., steerable or guidewire catheter embodiments) inserted using, for example, a transseptal or retrograde aortic approach into predetermined sections of the heart to ablate, in some instances, accessory pathways. The optical configurations configured with such a catheter embodiment, or any of the arrangements disclosed herein, can include commercial available optical elements, as known by those of ordinary skill in the art, or custom optical elements to deliver and/or collect predetermined light spectra from multiple locations about the distal end of such catheters.

When utilized with ablation catheter embodiments, catheter 22 can be advanced into the targeted region, wherein a designed ablation element (not shown) of catheter 22 can be energized by means known in the art so as to form, for example, a lesion 23 in the surrounding tissue 28. When utilized in such a manner, catheter 22 often includes one or more illumination fibers 26 (one shown for simplicity) and one or more collection fibers 24 (again one shown for simplicity), as shown in FIG. 2(a), running from about the distal end to the proximal end of catheter 22 so as to direct illumination wavelengths and collect desired radiation (as shown with directional arrows) respectively before, during or after application of ablation energy.

As a beneficial embodiment, predetermined illumination radiation of at least about 250 nm and up to about 1500 nm, but most often radiation from about 600 nm to about 970 nm, from one or more illumination fibers 26 configured about the distal end of catheter 22 is directed substantially along the same direction with catheter 22 (direction denoted by the letter Z and as shown with a directional arrow). Such directed radiation is received by tissue components, such as normal tissue, non-normal tissue, in addition to modified tissue components, such as lesion 23 along an emission cone angle of illumination fiber(s) 26 or with illumination intensities as produced by adapted enhancing optical elements, such as, but not limited to, micro-lenses, mirrors, graded-index lenses, diffractive optical elements and other fiber performance enhancing elements as known in the art so as to induce NIR elastic scattered light in a backscattered geometry.

Upon such backscattered produced radiation, the one or more collection fibers 24 configured with catheter 22, receives a predetermined portion of the induced NIR elastic light scattered radiation from probed tissue at a receiving point (denoted as P′ in FIG, 2(a)), laterally removed from the emitting point of the one or more illumination fibers 26, (denoted as P as shown in FIG. 2(a)). Such induced radiation is then directed by collection fiber(s) 24 to the spectral analysis and detector compartments as illustrated in FIGS. 1(a)-(c) as detailed above.

The detectors, as shown and discussed above with respect to FIGS. 1(a)-(c), transforms a photometric signal into an electrical signal. The electrical signal is captured by an electronic circuit (not shown) and is converted to a digital form with conventional analog/digital converters as known and understood by those skilled in the art. The digital signal is then digitally pre-processed by digital signal processing residing in, for example, analysis means 18, as shown in FIGS. 1(a)-(c), and information is stored in memory. The information can be accessed by analysis means 18, or by one or one or more additional external computing devices (not shown) for further analysis, and presented to users through a graphic user interface via designed or commercial software, as disclosed herein.

A surprising and unexpected result during ablation procedures is the characteristic changes in the received spectra, which enables the detection and determination of deleterious thermal effects (i.e., via intensity and/or characteristic changes in received spectra) resulting from charring, formation of steam pops, and coagulum. The operator can use such information to increase or decrease the energy delivered to the site so as to control the final depth of the lesion while preventing the observed thermal deleterious effects or terminate the ablation procedure altogether.

While such an arrangement, as shown in FIG. 2(a) is beneficial, it is to be appreciated that example fibers (i.e., fibers 24 and 26) used for directing desired radiation components can also be coupled external (not shown) to catheter 22. In such a non-coupled arrangement, fibers 24 and 26, are not directly targeting tissue 28 under catheter 22 and thus, such an arrangement is designed to record the presence on ablated tissue (e.g. lesion 23) as it expands in time outwards from the point of contact with ablation energizing element of catheter 22 and enables ease of operation by not having to overtly modify existing catheter embodiments. As a result, there is a delay time from the point of initiation of ablation to the time that ablation will be detected by the spectroscopic analysis methods of the present invention when using such an arrangement.

FIG. 2(b) shows a variation of the catheter embodiment of FIG. 2(a) and is generally designated as reference numeral 20′. Such an arrangement again can include various probes, such as, but not limited to, a catheter 22 utilized for ablation procedures and modified according to the descriptions presented herein. As illustrated, one or more fibers 30 can again be used for collection while one or more fibers 26 may be used to deliver the illumination. In this novel embodiment, however, one or more additional fibers 27 may be configured with catheter 22 to probe (i.e., illuminate) the tissue, such as a formed or a forming lesion 23 in the case where the catheter is used to ablate the tissue at an angle different than normal to the tissue's 28 surface. The presence of an additional collection fiber 31 not in contact with tissue 28 can also be added by modification to allow catheter embodiments, as shown by example in FIG. 2(b), to probe the formation of coagulum, steam pops, and/or charring in the area surrounding the catheter that is not in direct contact with tissue 28 and enable evaluation of the orientation of the catheter with respect to the tissue surface. An advanced example arrangement involves a plurality of fibers alternated as illumination and/or collection of scattered light in a predetermined sequence so as to enable even more accurate assessment of the characteristic of ablation and the surrounding catheter environment (formation of coagulum, steam pops, charring, etc.).

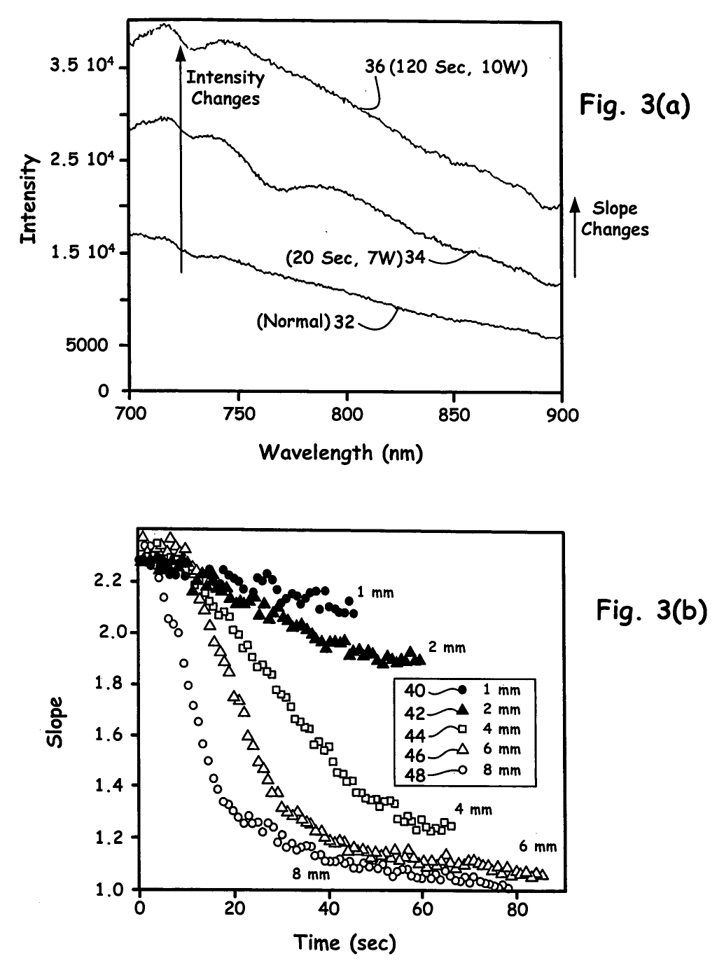

FIG. 3(a) shows experimental data of about a two-fold increase (denoted by the directional arrow) in the intensity of the backscattering light during tissue ablation. Such a result is exemplified with spectra from normal tissue 32 exposed to ablation powers of 7 W for 20 seconds 34 and subsequently 10 W for 120 seconds 36. Such a change in intensity can be utilized, as one example, to detect steam pop formation (micro bubbles) resulting from heating of the surrounding tissue fluids.

FIG. 3(a) also shows a changing slope of the spectral profile towards the longer wavelengths (i.e., at about the 900 nm range) (denoted by the shorter directional arrow) due to the ablation exposure times and deposited thermal energy.

FIG. 3(b) shows the slope of the spectrum of different sized lesions monitored during ablation lesion formation with different final depths. Thus, FIG. 3(b) shows the slope vs. time for 5 different ablations that resulted to lesions having depths of about 1 mm (40), 2 mm (42), 4 mm (44), 6 mm (46), and 8 mm (48). It is to be appreciated from this experimental data set that the different rates by which the slope is changing depends on the power settings of the catheter. From such data, one can extract the rate of tissue ablation since the slope is related to the depth of the lesion. This can be particular important for deeper lesions where direct measurement of the depth using the fibers may be impossible. More specifically, the measurement of the slope can provide accurate results for lesion depths of up to about 10 mm in human cardiac tissue. However, by measuring the rate of tissue ablation during the initial 6 mm, one can extrapolate the ablation time needed to create lesions of any depth.

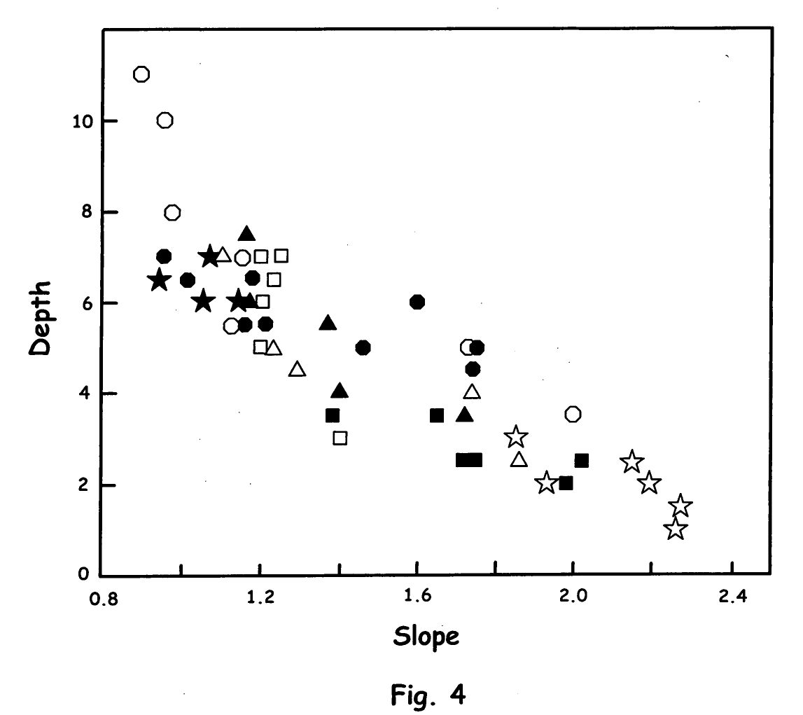

FIG. 4 illustrates the substantially linear relationship between depth and spectral profile using as a marker, the slope of the profile after a linear fit of the profile between 730 nm and 900 nm. To define an example measured slope, the ratio of the spectral intensity at 730 nm over that at 910 nm is plotted from predetermined spectra received from bovine heart tissue during an ablation procedure for a particular created lesion. Then additional slope values for different lesions created using different ablation times and power settings resulting in different lesion depths is added to the overall plot, as shown in FIG. 4. Accordingly, FIG. 4 summarizes experimental results showing the depth of the ablated tissue and the corresponding slope of the accompanying spectral profile. These results clearly indicate an almost linear relationship between these two parameters for lesion depths up to about 6 mm.

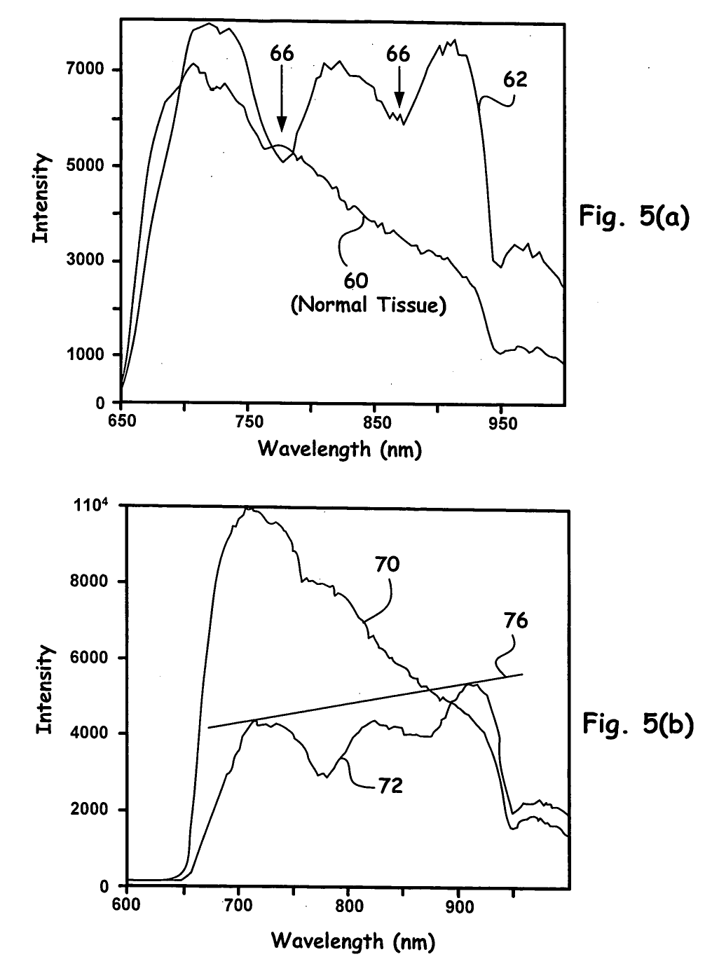

FIG. 5(a) illustrates the real-time detection of coagulum formation during catheter ablation treatment from the characteristic changes in the detected spectral profile while FIG. 5(b) illustrates the real-time detection of charring during catheter ablation treatment. Thus, FIG. 5(a) shows a normal tissue spectrum 60 and the presence of two spectral dips 66 in a received spectrum 62, indicating the presence of two absorption peaks associated with the presence of coagulum. FIG. 5(b) shows a spectrum of normal tissue 70 and a subsequent spectrum 72 in the presence of charring. From the results of FIG. 5(b), as utilized by the methods and various apparatus of the present invention, charring tends to exhibit intensities of the scattered light at 730 nm that is lower to that at 910 nm (i.e., for the spectral calibration used during this experiment). This leads to an example value of the estimated slope of less than 1. The absolute values of the slope shown above are somewhat arbitrary. This comes from the fact that the recorded spectra have not been corrected for instrument response nor for the spectral profile of the white light used for illumination. Therefore, although all trends and qualitative behaviors describe above are valid, the absolute values of the slopes and the relative intensities of the spectra at different wavelengths need to be adjusted to take into account instrument response and spectrum of input illumination light

Accordingly, the present invention utilizes primarily NIR light scattering to provide information about predetermined tissue properties prior to as well as during certain predetermined therapeutic procedures. In particular, with respect to ablation procedures, the present invention can provide information with regards to lesion formation, depth of penetration of the lesion, cross-sectional area of the lesion in the tissue, recognition of charring, recognition of the formation of coagulum, differentiation of ablated tissue from healthy, diseased, and/or abnormal tissue, and recognition of evaporate water in the blood and tissue leading to microbubbles (i.e., steam pop formation) is beneficially enabled.

Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The invention claimed is:

1. A spectroscopic method for real-time examination of biological tissue, comprising:
deploying a diagnostic and/or treatment tool on, in, or near a predetermined tissue site, wherein said tissue site comprises at least one tissue selected from: modified tissue, pre-treated tissue, and a tissue undergoing modification;
providing one or more predetermined optical conduits adapted to direct an interrogation radiation source at said predetermined tissue site and one or more predetermined optical conduits adapted to receive an induced predetermined backscattered radiation from said predetermined tissue site resulting from said directed interrogation radiation; and
measuring one or more elastic light scattering spectra resulting from said induced backscattered radiation to assess in real-time, an abnormal lesion, a lesion formation, a depth of penetration of said lesion, a cross-sectional area of said lesion in the tissue, recognition of charring, recognition of the formation of coagulum, differentiation of ablated tissue from healthy or abnormal tissue and/or recognition of evaporate water in the blood and tissue leading to steam pops.

2. The method of claim 1, wherein a relative increase in intensity and a red-shifted component of said measured spectra is utilized to real-time monitor said modified tissue and said tissue undergoing modification.

3. The method of claim 1, wherein a rate of one or more spectral changes resulting from said measured spectra enables extrapolation of an ablation depth of up to about 1 cm.

4. The method of claim 1, wherein a rate of one or more spectral changes resulting from said measured spectra enables extrapolation of the normal tissue.

5. The method of claim 1, wherein a rate of one or more spectral changes resulting from said measured spectra enables extrapolation of the normal tissue.

6. The method of claim 1, wherein one or more spectral changes of said measured spectra indicates the presence of abnormal tissue.

7. The method of claim 1, wherein a measured spectral change of said measured spectra is utilized to detect steam pops.

8. The method of claim 1, wherein said measured spectra comprises characteristic absorption regions so as to detect the formation of coagulum.

9. The method of claim 1, wherein said measured spectra comprises characteristic absorption regions so as to detect the formation of charring.

10. The method of claim 1, wherein said interrogation radiation source comprises a spectral region between about 600 nm and about 1500 nm.

11. The method of claim 1, wherein said backscattered radiation comprises a spectral region between about 600 nm and about 970 nm.

12. The method of claim 1, wherein said optical conduit comprises optical fibers and optical fiber bundles.

13. The method of claim 12, wherein said optical fibers and said optical fiber bundles comprises polarization maintaining fibers.

14. The method of claim 12, wherein said optical fibers and said optical fiber bundles not in direct contact with tissue are arranged at a predetermnined angle so as to probe the formation of coagulum, steam pops, and/or charring in the area surrounding a modified region.

15. The method of claim 12, wherein said optical fibers and said optical fiber bundles not in direct contact with tissue are arranged at a predetermined angle so as to evaluate a position of said diagnostic and/or treatment tool with respect to said tissue surface.

16. The method of claim 9, wherein said optical fibers and said optical fiber bundles comprises a plurality of fibers alternated as illumination and/or collection fibers of directed and scattered radiation in a predetermined sequence.

17. The method of claim 1, wherein said one or more optical conduits are arranged at a predetermined distance from said deployed treatment and/or diagnostic tool.

18. The method of claim 1, wherein said treatment and/or diagnostic tool comprises an endoscope.

19. The method of claim 1, wherein said treatment and/or diagnostic tool comprises an ablation catheter.

20. An apparatus for assessing tissue components, comprising:
a treatment and/or diagnostic tool;
one or more interrogation radiation sources having predetermined wavelengths;
one or more optical fibers disposed within said treatment and/or diagnostic tool for directing said interrogation radiation sources to one or more targeted tissue components and additionally adapted for receiving and directing an induced backscattered radiation resulting from said one or more interrogated and targeted tissue components;
a device adapted to record one or more spectra of said induced backscattered radiation from said tissue components; and
means for analyzing one or more spectral changes of said spectra so as to assess in real-time, an abnormal lesion, a lesion formation, a depth of penetration of said lesion, a cross-sectional area of said lesion in the tissue, recognition of charring, recognition of the formation of coagulum, differentiation of ablated tissue from healthy or abnormal tissue and/or recognition of evaporate water in the blood and tissue leading to steam pops.

21. The apparatus of claim 20, wherein a relative increase in intensity and a red-shifted component of said recorded spectra is utilized to real-time monitor one or more modified tissue components.

22. The apparatus of claim 20, wherein a rate of said spectral changes enables extrapolation of an ablation depth of up to about 1 cm.

23. The apparatus of claim 20, wherein a rate of one or more spectral changes resulting from said measured spectra enables extrapolation of the normal tissue.

24. The apparatus of claim 20, wherein a rate of one or more spectral changes resulting from said measured spectra enables extrapolation of the normal tissue.

25. The apparatus of claim 20, wherein said spectral changes comprises the presence of charring.

26. The apparatus of claim 20, wherein said spectral changes comprises the presence of steam pops.

27. The apparatus of claim 20, wherein said spectral changes comprises characteristic absorption regions so as to detect the formation of coagulum.

28. The apparatus of claim 20, wherein said interrogation radiation source comprises emission wavelengths of between about 600 nm and about 970 nm.

29. The apparatus of claim 20, wherein said interrogation radiation source comprises at least one source selected from: broadband sources, narrow-band spectrally stable light emitting diodes (LEDs), narrow-band fluorescence sources, laser sources, and tunable optical sources.

30. The apparatus of claim 20, wherein said backscattered radiation comprises a spectral region between about 600 nm and about 1500 nm.

31. The apparatus of claim 20, wherein said optical conduit comprises at least one optical conduit further comprising: optical fibers, optical fiber bundles, and optical light guides.

32. The apparatus of claim 31, wherein said optical fibers and said optical fiber bundles comprises polarization maintaining fibers.

33. The apparatus of claim 31, wherein said optical fibers, optical fiber bundles, and optical light guides not in direct contact with tissue are arranged at a predetermined angle so as to probe the formation of coagulum, steam pops, and/or charring in the area surrounding a modified region.

34. The apparatus of claim 31, wherein said optical fibers and said optical fiber bundles comprises a plurality of fibers alternated as illumination and/or collection fibers of directed and scattered radiation in a predetermined sequence.

35. The apparatus of claim 20, wherein said treatment and/or diagnostic tool comprises an endoscope.

36. The apparatus of claim 20, wherein said treatment and/or diagnostic tool comprises an ablation catheter.

37. The apparatus of claim 20, wherein said detector device comprises at least one detector devices selected from: a charged coupled devices (CCDs), photodiodes, photomultipliers, spectral analyzers, two-dimensional array detectors, and multi-array detectors.

38. The apparatus of claim 20, wherein said analyzing means further comprises at least one device selected from: a computer, a firmware, a CPU, a graphical user interface, a software program, and a field-programmable gate array.

39. The apparatus of claim 20, wherein said treatment and/or diagnostic tool can be positioned up to about 90 degrees from the normal with respect to said one or more targeted tissue components.
